(19) United States
(12) Patent Application Publication
(10) Pub. No.: US 2022/0334102 A1
(43) Pub. Date: Oct. 20, 2022

(54) EX VIVO LYMPH NODE AND USES THEREOF

(71) Applicants: Jason Paul Gleghorn, Lincoln University, PA (US); Ryan Mark Zurakowski, Newark, DE (US)

(72) Inventors: Jason Paul Gleghorn, Lincoln University, PA (US); Ryan Mark Zurakowski, Newark, DE (US)

(73) Assignee: University of Delaware, Newark, DE (US)

(21) Appl. No.: 17/641,254

(22) PCT Filed: Sep. 16, 2020
(86) PCT No.: PCT/US20/51001

§ 371 Date: Mar. 8, 2022

Related U.S. Application Data
(60) Provisional application No. 62/900,774, filed on Sep. 16, 2019.

(51) Int. Cl. G01N 33/50; C12N 5/078; A01N 1/02; C12N 5/0783; C12N 5/0781

(52) U.S. Cl. CPC G01N 33/5082; C12N 5/0651; A01N 1/0247; A01N 1/021; C12N 5/0636; C12N 5/0635

(57) ABSTRACT

The present invention provides an ex vivo lymph node is provided. The ex vivo lymph node comprises an intact lobule in a chamber connected to an afferent lymphatic vessel and an efferent lymphatic vessel. The intact lobule is perfused with a lymphatic fluid into the chamber via the afferent lymphatic vessel and out of the chamber via the efferent lymphatic vessel and perfused with a vascular fluid into the intact lobule via an endogenous artery and out of the intact lobule via an endogenous vein. Also provided is a method for preparing the ex vivo lymph node. Further provided are methods for screening for an agent capable of changing the ex vivo lymph node, producing T lymphocytes or B lymphocytes and determining immunoreactivity of the ex vivo lymph node.

### CROSS-REFERENCE TO RELATED APPLICATION

This application claims priority to U.S. Provisional Application No. 62/900,774, filed Sep. 16, 2019, and the contents of which are incorporated herein by reference in their entireties for all purposes.

### FIELD OF THE INVENTION

The invention relates to an ex vivo lymph node and preparation and uses thereof.

### BACKGROUND OF THE INVENTION

The lymph node is the primary site for antigen presentation and enrichment of the adaptive immune system resulting in maturation of both cellular and humoral immunity. When antigen-presenting cells (APCs) discover and process pathogens, they migrate to a nearby lymph node where they present foreign antigen to T lymphocytes. The activation and proliferation of antigen-specific T lymphocytes primes the adaptive immune system and establishes immunologic memory enabling a robust response to future infections. Within B cell follicles, antigen bound by antibodies adheres to follicular dendritic cells, which present them to B lymphocytes resulting in maturation of the humoral immune response. The lymph node is isolated by highly selective transport barriers. The post-arterial capillaries, venules, and high endothelial venules (HEVs) that perfuse the lymphoid lobule are not fenestrated and only allow active transport. The subcapsular sinus network allows passive transport between the fluid lymph and the lobule, but across a fibrous barrier that limits transport and excludes large particles. This selectivity results in poor drug penetrance. In addition to serving as the primary site for the initiation of the adaptive immune response, the lymph node also acts as a sanctuary site for invasive agents such as bacteria, cancer cells, and viruses. It is imperative to better understand cell trafficking and drug transport in the lymph node. However, many of the intricate and dynamic processes that occur in the lymph node remain largely under studied. This can be attributed to the fact that the current gold standard models of the lymph node have very little applicability to human immunity. Animal models have obvious advantages, such as an intact lymphatic system and normal physiologic conditions. However, these models and mechanisms are not directly transferrable to human immunity. Existing organ-on-a-chip models can include human cells or murine tissue sections. These types of models often ignore complex, important physiological components such as mechanical forces, 3D spatial organization, or intercellular interactions.

There remains a need for an ex vivo lymph node reproducing the appropriate microenvironment in an in vivo lymph node, resulting in physiologically plausible intercellular interactions.

### SUMMARY OF THE INVENTION

The present invention relates to an ex vivo lymph node in a long term culture with independent dynamic control of lymphatic perfusion and vascular perfusion suitable for studying trafficking of cells, especially immune cells, drugs, metabolites, blood, serum, and pathogens into and throughout the lymph node via the lymphatic and/or vascular circulation and the inflammation or immune responses in the lymph node. The inventors have developed a way to keep an ex vivo lymph node viable for days by perfusion of lymphatic and vascular vessels.

An ex vivo lymph node is provided. The ex vivo lymph node comprises an intact lobule in a chamber connected to an afferent lymphatic vessel and an efferent lymphatic vessel. The intact lobule retains viable endogenous cells in the intact lobule in vivo, and an endogenous artery and an endogenous vein each attached to the intact lobule in vivo when the intact lobule is removed from a donor. The intact lobule is perfused with a lymphatic fluid into the chamber via the afferent lymphatic vessel and out of the chamber via the efferent lymphatic vessel and perfused with a vascular fluid into the intact lobule via the endogenous artery and out of the intact lobule via the endogenous vein.

The ex vivo lymph node may further comprise an endogenous capsule enclosing the chamber and encompassing the intact lobule. The afferent lymphatic vessel may be an endogenous afferent lymphatic vessel attached to the endogenous capsule, and the efferent lymphatic vessel may be an endogenous efferent lymphatic vessel attached to the endogenous capsule in vivo when the intact lobule is removed from the donor. The ex vivo lymph node may further comprise an endogenous sinus adjacent to the intact lobule and encompassed by the endogenous capsule in vivo when the intact lobule is removed from the donor.

In the ex vivo lymph node, at least 80% of the endogenous cells may remain viable for at least 24 hours after the intact lobule is removed from the donor. The intact lobule may be perfused with the lymphatic fluid and the vascular fluid for at least 30 minutes. The lymphatic fluid may comprise an inlet lymphatic fluid flowing into the chamber from a lymphatic fluid reservoir via the afferent lymphatic vessel and an outlet lymphatic fluid flowing out of the chamber into an efferent collection via the efferent lymphatic vessel, and the inlet lymphatic fluid and the outlet lymphatic fluid have different components. The vascular fluid may comprise an inlet vascular fluid flowing into the intact lobule from a vascular fluid reservoir via the endogenous artery and an outlet vascular fluid flowing out of the lobule into a venous collection via the endogenous vein, and the inlet vascular fluid and the outlet vascular fluid have different components. The lymphatic fluid may flow under control by a lymphatic inlet pressure. The vascular fluid may flow under control by a vascular inlet pressure. The lymphatic fluid and the vascular fluid may flow independently.

According to the ex vivo lymph node, at least one of the afferent lymphatic vessel, the efferent lymphatic vessel, the endogenous artery and the endogenous vein may be connected directly to a cannulation device. An additional endogenous artery may be attached to the intact lobule and closed off. The intact lobule may be cultured.

The endogenous cells may comprise B lymphocytes, T lymphocytes, macrophages, dendritic cells, follicular dendritic cells, red blood cells or a combination thereof.

According to the ex vivo lymph node, the donor may be a human. The donor may have or be predisposed to a lymph node related disease or condition.

A method for preparing an ex vivo lymph node is also provided. The preparation method comprises providing an intact lobule in a chamber. The intact lobule retains viable endogenous cells in the intact lobule, and an endogenous artery and an endogenous vein each attached to the intact lobule in vivo when the intact lobule is removed from a donor. The chamber is connected to an afferent lymphatic vessel and an efferent lymphatic vessel. The preparation method also comprises perfusing the intact lobule with a lymphatic fluid flowing into the chamber via the afferent lymphatic vessel and out of the chamber via the efferent lymphatic vessel. The preparation method further comprises perfusing the intact lobule with a vascular fluid flowing into the intact lobule via the endogenous artery and out of the intact lobule via the endogenous vein.

The preparation method may further comprise enclosing the chamber and encompassing the intact lobule by an endogenous capsule, and the afferent lymphatic vessel may be an endogenous afferent lymphatic vessel attached to the endogenous capsule, and the efferent lymphatic vessel may be an endogenous efferent lymphatic vessel attached to the endogenous capsule in vivo when the intact lobule is removed from the donor. The endogenous capsule may further encompass an endogenous sinus adjacent to the intact lobule in vivo when the intact lobule is removed from the donor.

According to the preparation method, at least 80% of the endogenous cells may remain viable for at least 24 hours after the intact lobule is removed from the donor.

The preparation method may further comprise perfusing the intact lobule with the lymphatic fluid and the vascular fluid for at least 30 minutes.

The preparation method may further comprise flowing an inlet lymphatic fluid into the chamber from a lymphatic fluid reservoir via the afferent lymphatic vessel and flowing an outlet lymphatic fluid out of the chamber into an efferent collection via the efferent lymphatic vessel, and the inlet lymphatic fluid and the outlet lymphatic fluid may have different components. The preparation method may further comprise adjusting components of the inlet lymphatic fluid.

The preparation method may further comprise flowing an inlet vascular fluid into the intact lobule from a vascular fluid reservoir via the endogenous artery and flowing an outlet vascular fluid out of the lobule into a venous collection via the endogenous vein, and the inlet vascular fluid and the outlet vascular fluid may have different components. The preparation method may further comprise adjusting the components of the inlet vascular fluid. The preparation method may further comprise adjusting a lymphatic inlet pressure to control the flow rate of the lymphatic fluid.

The preparation method may further comprise a vascular inlet pressure to control the flow rate of the vascular fluid. The preparation method may further comprise connecting at least one of the afferent lymphatic vessel, the efferent lymphatic vessel, the endogenous artery and the endogenous vein directly to a cannulation device.

The preparation method may further comprise culturing the intact lobule.

For each preparation method, an ex vivo lymph node prepared according to the method is provided.

A method for screening for an agent capable of changing the ex vivo lymph node of the present invention is further provided. The screening method comprises exposing the ex vivo lymph node to a test agent; and monitoring a behavior of the intact lobule before and after the exposure. A change in the behavior after the exposure indicates that the test agent is capable of changing the ex vivo lymph node. The test agent may be selected from the group consisting of blood, serum, culture media, foreign cells, drugs, metabolites, analysis reagents, antigen, bacteria, parasites, viruses, antigen-loaded antigen-presenting cells, cytokines, and combinations thereof. The behavior of the intact lobule may be selected from the group consisting of cell viability, cell migration, metabolites, drug density, bulk transport rates, immunoreactivity, inflammation, follicle formation, and combinations thereof. The screening method may further comprise obtaining a lymphatic measurement. The screening method may further comprise obtaining a vascular measurement is obtained. The screening method may further comprise predicting an additional behavior of the intact lobule based on the lymphatic or vascular measurement.

A method for producing T lymphocytes is provided. The T lymphocyte production method comprises exposing the ex vivo lymph node of the present invention to a cognate antigen under conditions suitable for proliferation of cognate antigen specific T lymphocytes, whereby the number of the cognate antigen specific T lymphocytes increases at least three times after the exposure. The T lymphocyte production method further comprises analyzing the cognate antigen specific T lymphocytes.

A method for producing B lymphocytes is provided. The B lymphocyte production method comprises exposing the ex vivo lymph node of the present invention to an antigen under conditions suitable for proliferation of antigen specific B lymphocytes. As a result, the number of the antigen specific B lymphocytes increases at least three times after the exposure. The B lymphocyte production method may further comprise analyzing the antigen specific lymphocytes.

A method for determining immunoreactivity of the ex vivo lymph node of the present invention is provided. The immunoreactivity determination method comprises exposing the ex vivo lymph node to an antigen under conditions suitable for proliferation of antigen specific lymphocytes, and monitoring a behavior of the intact lobule before and after the exposure. A change in the behavior upon exposure indicates that the ex vivo lymph node is immunoreactive to the antigen. The antigen may be selected from the group consisting of bacterial peptides, viral peptides, fungal peptides, parasite peptides, tumor-specific peptides, foreign donor peptides, drugs and metabolites, endogenous molecules, and combinations thereof. The behavior of the lobule may be selected from the group consisting of formation of follicles, generation of activated and proliferating T or B lymphocytes, generation of differentiated B lymphocytes and/or plasma cells, generation of CD4 and/or CD8 T lymphocytes with an effector function, and a combination thereof.

### BRIEF DESCRIPTION OF THE DRAWINGS

FIGS. 1A-B illustrate that the LNChip enables whole lymph nodes to be cultured ex vivo. A) LNChip rendering of a prototype built and B) schematic outlining connections to the afferent lymphatic vessels, efferent lymphatic vessel, artery, and vein and the routes of perfusion to control fluid flows.

FIGS. 2A-D show successful cannulation of an afferent lymphatic vessel. A) 3D rendering and B-D) cannulation of an afferent lymphatic vessel in a human lymph node with a pulled glass needle and secured with sutures.

FIGS. 3A-D show successful culture of whole human lymph nodes. Human lymph nodes from transplant donors were cultured in ex vivo lymph node culture system. One afferent lymphatic vessels and the efferent lymphatic vessel and artery and vein were cannulated with a pulled glass needle as in FIG. 2 and cultured in a medium bath as depicted in FIG. 1. Lymph nodes were cultured for 48 hours connected and perfused with culture medium or cultured for 48 hours in culture medium within the device with no vessels cannulated nor perfused. Following culture, lymph nodes were cut with a scalpel and a commercial cell viability live-dead kit was used to stain cells en face on the cut surface. A) Live-dead images of a human lymph node with perfusion of a single afferent lymph node and culture for 48 hours compared to B) non-perfused. Green labeled cells represents live cells, and red labeled cells represent dead cells. The trend of widespread cell death in non-cannulated and non-perfused lymph nodes is also true after 24 hours of culture, demonstrating that the vascular and circulatory vessel networks must be perfused for successful culture. C, D) Perfused nodes demonstrate follicles within the lobules after culture via live dead (C) and H&E sections (D) demonstrating normal morphological structures.

FIGS. 4A-E show viable T lymphocytes following culture. Following 48 hours of culture (vessels cannulated and perfused) as described in FIG. 3, lymph nodes were serially sectioned and imaged using brightfield, and immunofluorescence methods. T cell depletion following 48 hours of perfused culture evident in A) brightfield and B) CD3 immunostained histological LN sections. Imaging LN lobule with C) Live/Dead D) DAPI and E) CD3 IHC, demonstrate viability of remaining T lymphocytes following 48 hours of ex vivo culture. Additionally, these data demonstrate the loss of lymphocytes from the lymph node, as is expected and normal in a functioning organ.

FIGS. 5A-C illustrate an intravital window culture device. A) Side view of device prototype and CAD rendering B) isometric view and C) exploded view.

### DETAILED DESCRIPTION OF THE INVENTION

The present invention relates to an ex vivo lymph node model and its preparation and applications. The inventors have successfully developed a long term culture of a lymph node from a donor containing a lobule with intact geometry as well as lymphatic and vascular (blood) vessels and networks, and have surprisingly discovered that the resulting ex vivo lymph node remains viable for at least 48 hours when perfused independently with a lymphatic fluid and a vascular fluid. The ex vivo lymph node remains viable for a sufficient duration to allow for drug distribution, lymphocyte migration and lymphocyte differentiation experiments, and allows continuous non-destructive sampling of venous output and lymph fluid.

A lymph node is a kidney-shaped organ enclosed by a capsule. The lymph node is divided into compartments known as lobules and filled with sinuses in the spaces not occupied by the lobules. The lobules are connected with blood vessels such as arteries and veins. Lymph enters the lymph node via afferent lymphatic vessels and leaves via efferent lymphatic vessels. Blood enters the lymph node via arteries and leaves via veins. The afferent and efferent lymphatic vessels are connected to the lymph node at the capsule. The arteries and veins are connected to the lymph node at the lobules. The inventors have successfully removed an entire or part of a lymph node from a donor and generated a viable ex vivo lymph node model mimicking an in vivo lymph node perfused with a lymphatic fluid via an afferent vessel and an efferent lymphatic vessel and perfused with a vascular fluid via an artery and a vein, and exhibiting a biological response, for example, inflammation or immune response, to test agents introduced through the lymphatic fluid and/or the vascular fluid.

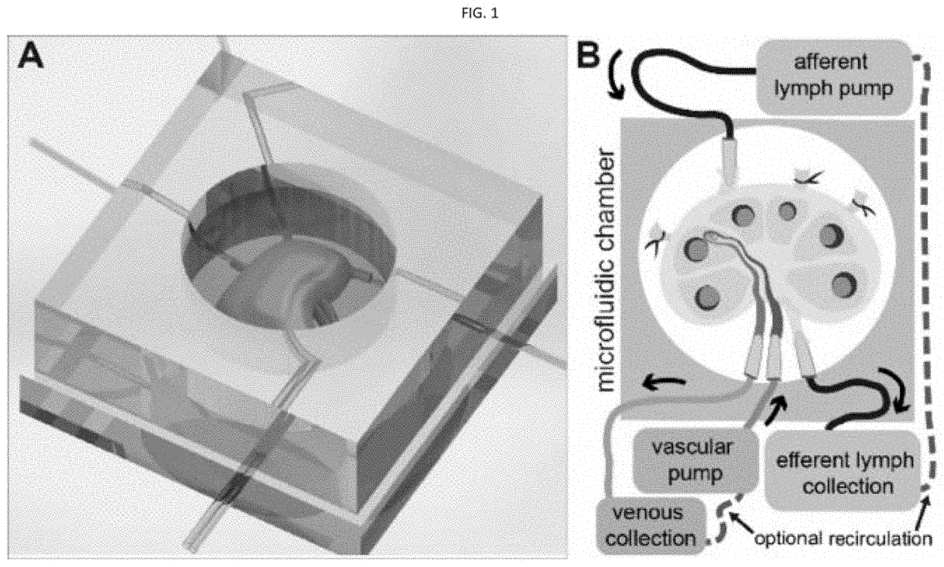

For example, as illustrated in FIG. 1, a lymph node chip (LNChip) is set up to enable a whole lymph node to be culture ex vivo in microfluidic chamber. The ex vivo lymph node is connected with two sets of vessels and perfused with (a) lymphatic fluid coming via a vessel connected with an afferent lymph pump and leaving via a vessel connected to an efferent lymph collection, and (b) vascular fluid coming from a vessel connected to a vascular pump and leaving via a vessel connected to a venous collection. The afferent lymph pump and the efferent lymph collection may be connected for optional recirculation. The vascular pump and the venous collection may be connected for optional recirculation. By culturing a microfluidically perfused ex vivo lymph node as illustrated in FIG. 1, the inventors have recapitulated and isolated these critical characteristics of lymph node transport.

The term “ex vivo lymph node” as used herein refers to an entire or part of a lymph node that is removed from a donor and remains viable for a predetermined duration. The predetermined duration may be at least 3, 6, 12, 18, 24, 36, 48, 72, 84 or 96 hours, or 1, 2, 3, 4, 5, 6, 7 or 8 weeks. The ex vivo lymph node may contain endogenous cells or tissues, i.e., cells (e.g., lymphocytes) or tissues (e.g., lobule, capsule and sinus) in the lymph node in vivo before the lymph node is removed from the donor in preparation of the ex vivo lymph node. The ex vivo lymph node may also contain foreign cells or tissues, i.e., cells or tissues not in the lymph node in vivo before the lymph node is removed from the donor in preparation of the ex vivo lymph node.

The term “viable” used herein refers to cells that are proliferating or a tissue or organ that contains cells that are proliferating and exhibits a biological activity. For example, a viable ex vivo lymph node contains proliferating lymphocytes and shows immunoreactivity upon exposure to a test agent.

The term “intact lobule” as used herein refers to a lobule retaining a substantial portion (e.g., at least 50%, 60%, 70%, 80%, 90%, 95%, 99% or 100%) of the cells (e.g., lymphocytes) and/or structure in or size (e.g., average diameter, volume or dimension) or shape of the lobule in a lymph node in vivo when the lymph node or a part thereof, including the lobule, is removed from a donor in preparation of an ex vivo lymph node. The intact lobule may retain a region of cortex with combined follicle B lymphocytes, a paracortex of T lymphocytes, and/or a part of the lobule in the medulla in the lobule in a lymph node in vivo when the lymph node or a part thereof, including the lobule, is removed from a donor in preparation of an ex vivo lymph node. The intact lobule may retain the artery and vein attached to the lobule in a lymph node in vivo when the lymph node or a part thereof, including the lobule, is removed from a donor in preparation of an ex vivo lymph node. The intact lobule may retain biological activities such as T cell expansion, lymphocyte programming and immunoreactivity.

The term “capsule” as used herein refers to a supporting meshwork formed by thin reticular fibers (reticulin) of a reticular connective tissue enclosing the ex vivo lymph node. The capsule may be endogenous, i.e., the capsule enclosing the lymph node in a lymph node in vivo when the lymph node or a part thereof, including the lobule, is removed from a donor in preparation of an ex vivo lymph node. The endogenous capsule may encompass one or more lobules and sinuses in the lymph node in vivo when the lymph node or a part thereof, including the lobule, is removed from a donor in preparation of an ex vivo lymph node, and may remain encompassing the lobules and/or sinuses in the ex vivo lymph node. The endogenous capsule may be connected with afferent or efferent lymphatic vessels in vivo when the lymph node or a part thereof, including the lobule, is removed from the donor in preparation of an ex vivo lymph node, and may remain connected with the vessels in the ex vivo lymph node.

The term “lymphatic fluid” used herein refers to a fluid flowing throughout an ex vivo lymph node via afferent and efferent lymphatic vessels. The lymphatic fluid may comprise lymphocytes, proteins, lipids, antigen presenting cells, antigen, infectious agents including bacteria and viruses, and pharmaceutical agents. The lymphatic fluid may flow into the ex vivo lymph node via an afferent lymphatic vessel and out of the ex vivo lymph node via an efferent lymphatic vessel. The lymphatic fluid flowing into the ex vivo lymph node is an inlet lymphatic fluid while the lymphatic fluid flowing out of the ex vivo lymph node is an outlet lymphatic fluid. The lymphatic fluid may be recirculated by connecting the efferent lymphatic vessel to the afferent lymphatic vessel.

The term “afferent lymphatic vessel” used herein refers to a tube or channel connected to an ex vivo lymph node and carrying a lymphatic fluid into the ex vivo lymph node. The afferent lymphatic vessel may be endogenous, i.e., the afferent lymphatic vessel connected to the capsule and enclosing the lymph node in vivo when the lymph node or a part thereof is removed from the donor in preparation of the ex vivo lymph node. The afferent lymphatic vessel may be foreign, i.e., not attached to the lymph node in vivo or not from the donor. The afferent lymphatic vessel may be synthetic. The afferent lymphatic vessel may be a needle made of, for example, pulled glass or metal, a microfluidic channel or a cannula.

The term “efferent lymphatic vessel” used herein refers to a tube or channel connected to an ex vivo lymph node and carrying a lymphatic fluid out of the ex vivo lymph node. The efferent lymphatic vessel may be endogenous, i.e., the efferent lymphatic vessel connected to the capsule and enclosing the lymph node in vivo when the lymph node or a part thereof is removed from the donor in preparation of the ex vivo lymph node. The efferent lymphatic vessel may be foreign, i.e., not attached to the lymph node in vivo or not from the donor. The efferent lymphatic vessel may be synthetic. The efferent lymphatic vessel may be a needle made of, for example, pulled glass or metal, a microfluidic channel or a cannula.

The term “lymphatic fluid reservoir” used herein refers to a device connected to an afferent lymphatic vessel, from which an inlet lymphatic fluid flows into an ex vivo lymph node via the afferent lymphatic vessel. The lymphatic fluid may be modified in the lymphatic fluid reservoir. For example, a test agent may be mixed with the lymphatic fluid in the lymphatic fluid reservoir so that the test agent may be introduced into the ex vivo lymph node via the inlet lymphatic fluid.

The term “efferent collection” used herein refers to a device connected to an efferent lymphatic vessel, into which an outlet lymphatic fluid flows out of an ex vivo lymph node via the afferent lymphatic vessel. The outlet lymphatic fluid in the efferent collection may be sampled and analyzed. The outlet lymphatic fluid may be characterized by measurements of its cells, biomarkers, drug concentrations, protein content, and virus and/or bacteria concentrations, and any soluble factor or nucleotide. Such lymphatic measurements may be used to guide the modification of the inlet lymphatic or vascular fluid.

The term “afferent lymph pump” used herein refers to any device (e.g. centrifugal, peristaltic, syringe) or mechanism (e.g., system to generate hydrostatic pressure head at an inlet relative to an outlet) that is able to move a lymphatic fluid into an ex vivo lymph node at, for example, lymph node sinus, through lymphatic vessels.

The term “afferent inlet pressure” used herein refers to the pressure of an inlet at an ex vivo lymph node that drives a lymphatic fluid into an ex vivo lymph node at, for example, lymph node sinus, through lymphatic vessels.

The term “vascular fluid” used herein refers to a fluid flowing throughout an ex vivo lymph node via an artery and a vein. The vascular fluid may comprise blood cells, serum, pharmaceutical agents, antigen, infectious agents including bacteria and/or viruses. The vascular fluid may flow into the ex vivo lymph node via an artery and out of the ex vivo lymph node via a vein. The vascular fluid flowing into the ex vivo lymph node is an inlet vascular fluid while the vascular fluid flowing out of the ex vivo lymph node is an outlet vascular fluid. The vascular fluid may be recirculated by connecting the vein to the artery, optionally via a vascular fluid reservoir.

The term “artery” used herein refers to a tube or channel connected to an ex vivo lymph node and carrying a vascular fluid into the ex vivo lymph node. The artery may be endogenous, i.e., the artery connected to a lobule in a lymph node in vivo when the lymph node or a part thereof, including the lobule, is removed from the donor in preparation of the ex vivo lymph node. The artery may be foreign, i.e., not attached to the lobule in the lymph node in vivo or not from the donor. The artery may be synthetic. The artery may be a needle made of, for example, pulled glass or metal, a microfluidic channel or a cannula.

The term “vein” used herein refers to a tube or channel connected to an ex vivo lymph node and carrying a vascular fluid out of the ex vivo lymph node. The vein may be endogenous, i.e., the vein connected to a lobule in a lymph node in vivo when the lymph node or a part thereof, including the lobule, is removed from the donor in preparation of the ex vivo lymph node. The vein may be foreign, i.e., not attached to the lobule in the lymph node in vivo or not from the donor. The vein may be synthetic. The vein may be a needle made of, for example, pulled glass or metal, a microfluidic channel or a cannula.

The term “vascular fluid reservoir” used herein refers to a device connected to an artery, from which an inlet vascular fluid flows into an ex vivo lymph node via the artery. The vascular fluid may be modified in the vascular fluid reservoir. For example, a test agent may be mixed with the vascular fluid in the vascular fluid reservoir so that the test agent may be introduced into the ex vivo lymph node via the inlet vascular fluid.

The term “venous collection” used herein refers to a device connected to a vein, into which an outlet vascular fluid flows out of an ex vivo lymph node via the vein. The outlet vascular fluid in the venous collection may be sampled and analyzed. The outlet vascular fluid may be characterized by measurements of its cells, biomarkers, drug concentrations, protein content, and virus and/or bacteria concentrations. Such vascular measurements may be used to guide the modification of the inlet vascular or lymphatic fluid.

The term “vascular pump” used herein refers to any device (e.g. centrifugal, peristaltic, syringe) or mechanism (e.g., system to generate hydrostatic pressure head at the inlet relative to the outlet) that is able to move a vascular fluid into an ex vivo lymph node through an artery or arterial connection.

The term “vascular inlet pressure” used herein refers to the pressure of an inlet that drives a vascular fluid into an ex vivo lymph node through an artery or arterial circulation.

The term “arterial connection” used herein refers to the connection to a large or small artery, arteriole, or capillary to allow fluid input (vascular fluid) into the vascular circulation.

The term “chamber” used herein refers to a space in which an intact lobule of an ex vivo lymph node is located. The chamber may be connected with an afferent lymphatic vessel and an efferent lymphatic vessel. The chamber may be filled, fully or partially, with a lymphatic fluid. The lymphatic fluid may flow into the chamber via the afferent lymphatic vessel and out of the chamber via the efferent lymphatic vessel. The chamber may be enclosed by a capsule, and the afferent lymphatic vessel and efferent lymphatic vessel may be attached to the capsule. The intact lobule may be covered or submerged by the lymphatic fluid in the chamber.

The invention provides an ex vivo lymph node. The ex vivo lymph node comprises a lymph node or a part thereof, including an intact lobule, in a chamber. The chamber is connected to an afferent lymphatic vessel and an efferent lymphatic vessel. The lymph node or a part thereof, including the intact lobule, is removed from a donor. The lymph node or a part thereof, including the intact lobule, retains viable endogenous cells in the lymph node or a part thereof, including the intact lobule, in vivo when the lymph node or a part thereof, including the intact lobule, is removed from the donor. The lymph node or a part thereof, including the intact lobule, is attached to an artery and a vein. The lymph node or a part thereof, including the intact lobule, is perfused with a lymphatic fluid into the chamber via the afferent lymphatic vessel and out of the chamber via the efferent lymphatic vessel. The lymph node or a part thereof, including the intact lobule, is perfused with a vascular fluid into the intact lobule via the artery and out of the intact lobule via the vein. The artery may be an endogenous artery attached to the intact lobule in vivo when the lymph node or a part thereof, including the intact lobule, is removed from the donor. The vein may be an endogenous vein attached to the intact lobule in vivo when the lymph node or a part thereof, including the intact lobule, is removed from the donor.

In one embodiment, the ex vivo lymph node comprises an intact lobule in a chamber. The chamber is connected to an afferent lymphatic vessel and an efferent lymphatic vessel. The intact lobule is removed from a donor. The intact lobule retains viable endogenous cells in the intact lobule in vivo when the intact lobule is removed from the donor. The intact lobule is attached to an artery and a vein. The intact lobule is perfused with a lymphatic fluid into the chamber via the afferent lymphatic vessel and out of the chamber via the efferent lymphatic vessel. The intact lobule is perfused with a vascular fluid into the intact lobule via the artery and out of the intact lobule via the vein. The artery is an endogenous artery attached to the intact lobule in vivo when the intact lobule is removed from the donor. The vein is an endogenous vein attached to the intact lobule in vivo when the intact lobule is removed from the donor.

The ex vivo lymph node may further comprise a capsule enclosing the chamber and encompassing the intact lobule. The capsule may be an endogenous capsule enclosing the lymph node and encompassing the intact lobule in vivo when the lymph node or a part thereof, including the intact lobule, is removed from the donor. The afferent lymphatic vessel may be an endogenous afferent lymphatic vessel attached to the endogenous capsule in vivo when the lymph node or a part thereof, including the intact lobule, is removed from the donor. The efferent lymphatic vessel may be an endogenous efferent lymphatic vessel attached to the endogenous capsule in vivo when the lymph node or a part thereof, including the intact lobule, is removed from the donor. For example, the ex vivo lymph node may further comprise an endogenous capsule enclosing the chamber and encompassing the intact lobule, and the afferent lymphatic vessel and the efferent lymphatic vessel may be endogenous afferent and efferent lymphatic vessels, respectively, attached to the endogenous capsule in vivo when the intact lobule is removed from the donor.

The ex vivo lymph node may further comprise an endogenous sinus. The endogenous sinus may be adjacent to the intact lobule in vivo when the lymph node or a part thereof, including the intact lobule, is removed from the donor. The endogenous sinus may be encompassed by the endogenous capsule in vivo when the lymph node or a part thereof, including the intact lobule, is removed from the donor. For example, the ex vivo lymph node may further comprise an endogenous sinus, which adjacent to the intact lobule in vivo or encompassed by the endogenous capsule in vivo when the intact lobule is removed from the donor.

The ex vivo lymph node may be viable for a predetermined duration. In the ex vivo lymph node, a substantial amount (e.g., at least 50%, 60%, 70%, 80%, 90%, 95%, 99% or 100%) of the endogenous cells remain viable for a predetermined duration. The predetermined duration may be at least 3, 6, 12, 18, 24, 36, 48, 72, 84 or 96 hours, or 1, 2, 3, 4, 5, 6, 7 or 8 weeks after the lymph node or a part thereof, including an intact lobule, is removed from the donor. In one embodiment, at least 50%, 60%, 70%, 80%, 90%, 95%, 99% or 100% of the endogenous cells remain viable for at least 24 hours after the intact lobule is removed from the donor.

The lymph node or a part thereof, including an intact lobule, may be perfused with the lymphatic fluid and the vascular fluid for at least 10, 20, 30, 45, 60, 90 or 120 minutes, or 6, 9, 12, 18, 24, 36, 48, 60, 72, 84 or 96 hours, or 1, 2, 3, 4, 5, 6, 7 or 8 weeks. For example, the intact lobule may be perfused with the lymphatic fluid and the vascular fluid for at least 10, 20, 30, 45, 60, 90 or 120 minutes, or 6, 9, 12, 18, 24, 36, 48, 60, 72, 84 or 96 hours, or 1, 2, 3, 4, 5, 6, 7 or 8 weeks.

The lymphatic fluid may comprise an inlet lymphatic fluid and an outlet lymphatic fluid. The inlet lymphatic fluid may flow into the chamber from a lymphatic fluid reservoir via the afferent lymphatic vessel. The outlet lymphatic fluid may flow out of the chamber into an efferent collection via the efferent lymphatic vessel. The inlet lymphatic fluid and the outlet lymphatic fluid may have different components. For example, the inlet lymphatic fluid may comprise a test antigen and the outlet lymphatic fluid may comprise more T lymphocytes specific for the test antigen than the inlet lymphatic fluid.

The vascular fluid may comprise an inlet vascular fluid and an outlet vascular fluid. The inlet vascular fluid may flow into the intact lobule from a vascular fluid reservoir via the endogenous artery. The outlet vascular fluid may flow out of the lobule into a venous collection via the endogenous vein. The inlet vascular fluid and the outlet vascular fluid may have different components. For example, the inlet vascular fluid may comprise a test drug and the outlet vascular fluid may comprise an elevated level of a metabolite of the drug than the inlet vascular fluid.

The lymphatic fluid may flow under control by a lymphatic inlet pressure. The flow rate of the lymphatic fluid may be increased, decreased or maintained by adjusting the lymphatic inlet pressure. The lymphatic inlet pressure may be generated by an afferent lymph pump connected to the afferent lymphatic vessel directly or indirectly. For example, a cannulation device may connect the afferent lymph pump with the afferent lymphatic vessel. The lymphatic inlet pressure may be a hydrostatic pressure generated by, for example, changing the height of the lymphatic fluid reservoir relative to the height of the efferent collection. The cannulation device may be a needle made of, for example, pulled glass or metal, a microfluidic channel or a cannula.

The vascular fluid may flow under control by a vascular inlet pressure. The flow rate of the vascular fluid may be increased, decreased or maintained by adjusting the vascular inlet pressure. The vascular inlet pressure may be generated by a vascular pump connected to the endogenous artery directly or indirectly. For example, a cannulation device may connect the vascular pump with the endogenous artery. The vascular inlet pressure may be a hydrostatic pressure generated by, for example, changing the height of the vascular fluid reservoir relative to the height of the venous collection. The cannulation device may be a needle made of, for example, pulled glass or metal, a microfluidic channel or a cannula.

The lymphatic fluid and the vascular fluid may flow independently. The lymphatic fluid and the vascular fluid may flow in separate pathways without crossover. The lymphatic fluid may be modified based on the characteristics of the vascular fluid, for example, a vascular measurement of the outlet vascular fluid. Likewise, the vascular fluid may be modified based on the characteristics of the lymphatic fluid, for example, a lymphatic measurement of the outlet lymphatic fluid.

At least one of the afferent lymphatic vessel, the efferent lymphatic vessel, the artery and the vein is connected directly to a cannulation device. The cannulation device may be a needle made of, for example, pulled glass or metal, a microfluidic channel or a cannula.

An additional endogenous vessel, for example, an artery, vein, afferent lymphatic vessel or efferent lymphatic vessel, may be attached to the lymph node or a part thereof, including an intact lobule, in the ex vivo lymph node and closed off in order to prevent leakage from the lymph node or a part thereof. In one embodiment, an additional endogenous artery, vein, afferent lymphatic vessel or efferent lymphatic vessel is attached to the intact lobule in the ex vivo lymph node and closed off. In another embodiment, an additional endogenous afferent lymphatic vessel is attached to the intact lobule in the ex vivo lymph node and closed off.

The lymph node or a part thereof, including an intact lobule, may be cultured under conditions suitable for maintaining the lymph node or a part thereof viable. For example, the lymph node or a part thereof may be cultured at a temperature above freezing point but not higher than 40° C., for example, at 37-39° C. (e.g., 38° C.), 22-28° C. (e.g., 25° C.), or 1-4° C. The lymph node or a part thereof may be cultured at 4-6% CO₂ (e.g., 5% CO₂). The lymph node or a part thereof may be cultured at a pH of less than 6, for example, an acidic pH for allergy studies.

The endogenous cells may comprise B lymphocytes, T lymphocytes, macrophages, dendritic cells, follicular dendritic cells, red blood cells or a combination thereof. In one embodiment, the endogenous cells comprise T lymphocytes.

The donor may be selected from the group consisting small animal models (e.g. mice, rats, rabbits), large animal models (e.g. goats, pig, horses, cows), human and non-human primates]. In one embodiment, the donor is a human. The donor may be healthy. The donor may have a lymph node related disease or condition. The donor may be predisposed to a lymph node related disease or condition. The lymph node related disease or condition may be selected from the group consisting of bacterial infection, viral infection, parasitic infection cancer, autoimmune disease, graft-versus-host or host-vs-graft disease, allergy, and lymphedema.

The present invention also provides a method for preparing an ex vivo lymph node. The preparation method comprises providing a lymph node or a part thereof, including an intact lobule, in a chamber. The chamber is connected to an afferent lymphatic vessel and an efferent lymphatic vessel. The lymph node or a part thereof, including the intact lobule, is removed from a donor. The lymph node or a part thereof, including the intact lobule, retains viable endogenous cells in the lymph node or a part thereof, including the intact lobule, in vivo when the lymph node or a part thereof, including the intact lobule, is removed from the donor. The lymph node or a part thereof, including the intact lobule, is attached to an artery and a vein. The preparation method also comprises perfusing the lymph node or a part thereof, including the intact lobule, with a lymphatic fluid into the chamber via the afferent lymphatic vessel and out of the chamber via the efferent lymphatic vessel. The preparation method further comprises perfusing the lymph node or a part thereof, including the intact lobule, with a vascular fluid flowing into the intact lobule via the endogenous artery and out of the intact lobule via the endogenous vein. The artery may be an endogenous artery attached to the intact lobule in vivo when the lymph node or a part thereof, including the intact lobule, is removed from the donor. The vein may be an endogenous vein attached to the intact lobule in vivo when the lymph node or a part thereof, including the intact lobule, is removed from the donor.

In one embodiment, the preparation method comprises providing an intact lobule in a chamber. The chamber is connected to an afferent lymphatic vessel and an efferent lymphatic vessel. The intact lobule is removed from a donor. The intact lobule retains viable endogenous cells in the intact lobule in vivo when the intact lobule is removed from the donor. The intact lobule is attached to an artery and a vein. The preparation method also comprises perfusing the intact lobule with a lymphatic fluid into the chamber via the afferent lymphatic vessel and out of the chamber via the efferent lymphatic vessel. The preparation method further comprises perfusing the intact lobule with a vascular fluid flowing into the intact lobule via the endogenous artery and out of the intact lobule via the endogenous vein. The artery is an endogenous artery attached to the intact lobule in vivo when the intact lobule is removed from the donor. The vein is an endogenous vein attached to the intact lobule in vivo when the intact lobule is removed from the donor.

The preparation method may further comprise enclosing the chamber and encompassing the intact lobule by an endogenous capsule. The capsule may be an endogenous capsule enclosing the lymph node and encompassing the intact lobule in vivo when the lymph node or a part thereof, including the intact lobule, is removed from the donor. The afferent lymphatic vessel may be an endogenous afferent lymphatic vessel attached to the endogenous capsule in vivo when the lymph node or a part thereof, including the intact lobule, is removed from the donor. The efferent lymphatic vessel may be an endogenous efferent lymphatic vessel attached to the endogenous capsule in vivo when the lymph node or a part thereof, including the intact lobule, is removed from the donor. In one embodiment, the preparation method further comprises enclosing the chamber and encompassing the intact lobule by an endogenous capsule, and the afferent lymphatic vessel and the efferent lymphatic vessel are endogenous afferent and efferent lymphatic vessels, respectively, attached to the endogenous capsule in vivo when the intact lobule is removed from the donor.

The preparation method may further comprise encompassing an endogenous sinus by the capsule. The endogenous sinus may be adjacent to the intact lobule in vivo when the lymph node or a part thereof, including the intact lobule, is removed from the donor. The endogenous sinus may be encompassed by the endogenous capsule in vivo when the lymph node or a part thereof, including the intact lobule, is removed from the donor. For example, the ex vivo lymph node may further comprise an endogenous sinus, which adjacent to the intact lobule in vivo or encompassed by the endogenous capsule in vivo when the intact lobule is removed from the donor.

According to the preparation method of the present invention, the ex vivo lymph node may be viable for a predetermined duration. A substantial amount (e.g., at least 50%, 60%, 70%, 80%, 90%, 95%, 99% or 100%) of the endogenous cells may remain viable for a predetermined duration. The predetermined duration may be at least 3, 6, 12, 18, 24, 36, 48, 72, 84 or 96 hours, or 1, 2, 3, 4, 5, 6, 7 or 8 weeks after the lymph node or a part thereof, including an intact lobule, is removed from the donor. In one embodiment, at least 50%, 60%, 70%, 80%, 90%, 95%, 99% or 100% of the endogenous cells remain viable for at least 24 hours after the intact lobule is removed from the donor.

The preparation method may further comprise perfusing the lymph node or a part thereof, including an intact lobule, with the lymphatic fluid and the vascular fluid for at least 10, 20, 30, 45, 60, 90 or 120 minutes, or 6, 9, 12, 18, 24, 36, 48, 60, 72, 84 or 96 hours, or 1, 2, 3, 4, 5, 6, 7 or 8 weeks. For example, the preparation method may further comprise perfusing the intact lobule with the lymphatic fluid and the vascular fluid for at least 10, 20, 30, 45, 60, 90 or 120 minutes, or 6, 9, 12, 18, 24, 36, 48, 60, 72, 84 or 96 hours, or 1, 2, 3, 4, 5, 6, 7 or 8 weeks.

The preparation method may further comprise flowing an inlet lymphatic fluid into the chamber from a lymphatic fluid reservoir via the afferent lymphatic vessel and flowing an outlet lymphatic fluid out of the chamber into an efferent collection via the efferent lymphatic vessel. The inlet lymphatic fluid and the outlet lymphatic fluid may have different components. For example, the inlet lymphatic fluid may comprise a test antigen and the outlet lymphatic fluid may comprise more T lymphocytes specific for the test antigen than the inlet lymphatic fluid. The preparation method may further comprise adjusting components of the inlet lymphatic fluid. The adjustment may be based on behavior of the intact lobule and/or components of the outlet lymphatic fluid.

The preparation method may further comprise flowing an inlet vascular fluid into the lymph node or a part thereof, including an intact lobule, from a vascular fluid reservoir via the endogenous artery and flowing an outlet vascular fluid out of the lymph node or a part thereof, including an intact lobule, into a venous collection via the endogenous vein. The inlet vascular fluid and the outlet vascular fluid may have different components. For example, the inlet vascular fluid may comprise a test drug and the outlet vascular fluid may comprise an elevated level of a metabolite of the drug than the inlet vascular fluid. The preparation method may further comprise adjusting components of the inlet vascular fluid. The adjustment may be based on behavior of the intact lobule and/or components of the outlet vascular fluid.

The preparation method may further comprise adjusting a lymphatic inlet pressure to control the flow rate of the lymphatic fluid. The flow rate of the lymphatic fluid may be increased, decreased or maintained by adjusting the lymphatic inlet pressure. The preparation method may further comprise generating the lymphatic inlet pressure by an afferent lymph pump connected to the afferent lymphatic vessel directly or indirectly. A cannulation device may connect the afferent lymph pump with the afferent lymphatic vessel. Where the lymphatic inlet pressure is a hydrostatic pressure, the hydrostatic pressure may be adjusted by changing the height of the lymphatic fluid reservoir relative to the height of the efferent collection. The cannulation device may be a needle made of, for example, pulled glass or metal, a microfluidic channel or a cannula.

The preparation method may further comprise adjusting a vascular inlet pressure to control the flow rate of the vascular fluid. The flow rate of the vascular fluid may be increased, decreased or maintained by adjusting the vascular inlet pressure. The preparation method may further comprise generating the vascular inlet pressure by a vascular pump connected to the artery directly or indirectly. A cannulation device may connect the vascular pump with the artery. Where the vascular inlet pressure is a hydrostatic pressure, the hydrostatic pressure may be adjusted by changing the height of the vascular fluid reservoir relative to the height of the venous collection. The cannulation device may be a needle made of, for example, pulled glass or metal, a microfluidic channel or a cannula.

According to the preparation method of the present invention, the lymphatic fluid and the vascular fluid may flow independently. The preparation method may further comprise flowing the lymphatic fluid and the vascular fluid in separate pathways without crossover. The preparation method may further comprise modifying the lymphatic fluid based on the characteristics of the vascular fluid, for example, a vascular measurement of the outlet vascular fluid. The preparation method may further comprise modifying the vascular fluid based on the characteristics of the lymphatic fluid, for example, a lymphatic measurement of the outlet lymphatic fluid.

The preparation method may further comprise connecting at least one of the afferent lymphatic vessel, the efferent lymphatic vessel, the artery and the vein directly to a cannulation device. The cannulation device may be a needle made of, for example, pulled glass or metal, a microfluidic channel or a cannula.

Where the lymph node or a part thereof, including an intact lobule, is attached to an additional endogenous vessel, for example, an artery, vein, afferent lymphatic vessel or efferent lymphatic vessel, the preparation method may further comprise closing off the additional endogenous vessel. In one embodiment, where the intact lobule in the ex vivo lymph node is attached to an additional endogenous vessel, for example, an additional endogenous artery, vein, afferent lymphatic vessel or efferent lymphatic vessel, the preparation method further comprises closing off the additional endogenous vessel. In another embodiment, where the intact lobule in the ex vivo lymph node is attached to an additional endogenous afferent lymphatic vessel, the preparation method further comprises closing off the additional endogenous afferent lymphatic vessel.

The preparation method may further comprise culturing the lymph node or a part thereof, including an intact lobule, under conditions suitable for maintaining the lymph node or a part thereof viable. For example, the preparation method may comprise culturing the lymph node or a part thereof at a temperature above freezing point but not higher than 40° C., for example, at 37-39° C. (e.g., 38° C.), 22-28° C. (e.g., 25° C.), or 1-4° C. The preparation method may comprise culturing the lymph node or a part thereof at 4-6% CO₂ (e.g., 5% CO₂). The preparation method may comprise culturing the lymph node or a part thereof at a pH of less than 6, for example, an acidic pH for allergy studies.

For each preparation method of the present invention, an ex vivo lymph node as prepared is provided.

The present invention further provides a method for screening for an agent capable of changing the ex vivo lymph node of the present invention. The screening method comprises exposing the ex vivo lymph node to a test agent and monitoring a behavior of the intact lobule in the ex vivo lymph node before and after the exposure. A change in the behavior after the exposure indicates that the test agent is capable of changing the ex vivo lymph node.

The test agent may be introduced to the ex vivo lymph node via the lymphatic fluid, vascular fluid or both. The test agent may be selected from the group consisting of blood, serum, culture media, foreign cells (e.g., lymphocytes), drugs (e.g., antiretroviral or chemotherapeutic drugs), metabolites, analysis reagents, antigen, bacteria, parasites, viruses, antigen-loaded antigen-presenting cells, cytokines, and combinations thereof. Every species of the test agent may be labeled by standard techniques, for example, staining of cells, radiomarkers on drugs and other molecules.

The behavior of the intact lobule may be selected from the group consisting of cell viability, cell migration, metabolites, drug density, bulk transport rates, immunoreactivity, inflammation, follicle formation, and combinations thereof. The drug density may be a concentration of the drugs achieved in various internal compartments of the lymph node, for example, the intact lobule. The bulk transport rate may be an average rate of cell or drug transport across the vascular and sinus barriers into or out of the lobule, expressed per unit of shared surface area of the barrier.

The screening method may further comprise obtaining a lymphatic measurement. Exemplary lymphatic measurements include counting cell emigration rates via flow cytometry, analyzing drug concentrations via mass spectrometry, and analyzing emigrant cell phenotype via fluorescent immunohistochemistry. The screening method may further comprise obtaining a vascular measurement. Exemplary vascular measurements include counting cell emigration rates via flow cytometry, analyzing drug concentrations via mass spectrometry, and analyzing emigrant cell phenotype via fluorescent immunohistochemistry. The screening method may further comprise predicting an additional behavior of the intact lobule based on the lymphatic or vascular measurement. For example, the prediction may be made using Finite Volume Method models that are solved using implicit variable-step/variable-order algorithms for stiff systems, identified using Maximum Likelihood methods.

The present invention further provides a method for producing T lymphocytes. The T lymphocyte production method comprises exposing the ex vivo lymph node of the present invention to a cognate antigen under conditions suitable for proliferation of cognate antigen specific T lymphocytes. As a result, the number of the cognate antigen specific T lymphocytes increases at least three times after the exposure. The cognate antigen may be introduced to the ex vivo lymph node via the lymphatic fluid, vascular fluid or both. The cognate antigen may be loaded on antigen presenting cells. The cognate antigen may be introduced with supporting cytokines. Exemplary supporting cytokines include IL-2, IL-7, and IL-12. The T lymphocyte production method may further comprise analyzing the cognate antigen specific T lymphocytes. For example, the cognate antigen specific T lymphocytes may be analyzed for its activated phenotype.

The present invention further provides a method for producing B lymphocytes. The B lymphocyte production method comprises exposing the ex vivo lymph node of the present invention to an antigen under conditions suitable for proliferation of antigen specific B lymphocytes. As a result, the number of the antigen specific B lymphocytes increases at least three times after the exposure. The antigen may be introduced to the ex vivo lymph node via the lymphatic fluid, vascular fluid or both. The antigen may be introduced with supporting cytokines. Exemplary supporting cytokines include IL-1, IL-4, IL-6, IL-7, and IL-10. The B lymphocyte production method may further comprise analyzing the antigen specific B lymphocytes. For example, the antigen specific B lymphocytes may be analyzed for its activated phenotype.

The present invention further provides a method for determining immunoreactivity of the ex vivo lymph node of the present invention. The immunoreactivity determination method comprises exposing the ex vivo lymph node to an antigen under conditions suitable for proliferation of antigen specific lymphocytes, and monitoring a behavior of the intact lobule before and after the exposure. A change in the behavior upon exposure indicates that the ex vivo lymphocyte is immunoreactive to the antigen. The antigen may be introduced to the ex vivo lymph node via the lymphatic fluid, vascular fluid or both. The antigen may be selected from the group consisting of bacterial peptides, viral peptides, fungal peptides, parasite peptides, tumor-specific peptides, foreign donor peptides, drugs and metabolites, endogenous molecules, and combinations thereof. The metabolites may be used to test allergy. The endogenous molecules may be used to test for autoimmune disorders. The behavior of the lobule may be selected from the group consisting of formation of follicles, generation of activated and proliferating T or B lymphocytes, generation of differentiated B lymphocytes and/or plasma cells, generation of CD4 and/or CD8 T lymphocytes with an effector function, and a combination thereof.

### Example 1. Long-Term Ex Vivo Culture of a Human Lymph Node

There is a critical need for the long term ex vivo culture of a human lymph node to answer mechanistic questions surrounding cell-cell interaction, cell trafficking, and drug transport. A novel microfluidic platform has been developed for a whole human lymph node with connections to the afferent and efferent lymphatic vessels along with the vein and artery. This system is a complete physiologically intact lymph node chip (FIG. 1). It is critical that the spatial organization of the immune cells is maintained throughout culture. T lymphocytes and dendritic cells reside mostly in the paracortex, whereas B-cells, follicular dendritic cells, and subcapsular macrophages reside in the cortex. This orientation is essential for antigen presentation and immune function in the lymph node. We have applied cannulation techniques to the human lymph node. By controlling the input and output flows of the organ, fluid flow and composition can be controlled selectively and dynamically through the vascular and lymphatic circulations and allow the organ to remain viable ex vivo.

Design Overview: A microfluidic platform (hLNChip) has been created for the ex vivo culture of a human lymph node with dynamically perfusable vascular and lymphatic networks for at least two (2) days of culture. This work has demonstrated successful ex vivo lymph node culture for 48 hours. Rigorous assessment and quantification of organ health have been determined with minimally invasive longitudinal measures and end-point assays. These data have been coupled with a computational model of transport in the lymph node to create a framework for non-invasive monitoring of organ health from temporal analyte concentrations in the efferent lymphatic and venous flows.

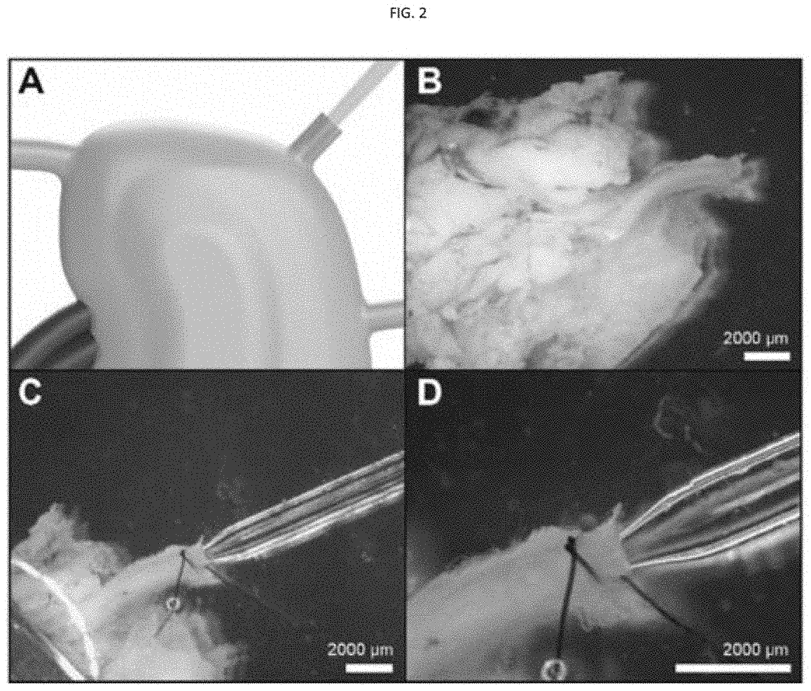

Lymph node dissection: The lymph nodes are embedded within fat and connective tissue proximal to internal organs. This fat pad has been treated identically to organs harvested for transplant. Immediately following surgical excision of the fat pad in the operating room, the proximal artery was cannulated and perfused with an anti-coagulation preservative solution (UW Viaspan) used for transplants to protect the organs for chilling and transport. The entire fat pad was placed on ice and transported to the University of Delaware. Microdissection on a sterescope in chilled UW Viaspan was performed to dissect out individual lymph nodes, remove excess surrounding fat, and mobilize the afferent and efferent lymphatic and vascular vessels for cannulation (FIG. 2).

hLNChip device fabrication and lymph node culture: hLNChip devices were created using standard materials and methods for microfluidic device fabrication. Briefly, a one centimeter thick slab of polydimethyl siloxane (PDMS) was cured in a dish and a circular hole punch (3 cm diameter) was used to create a hole in the PDMS. The PDMS was then bonded to a large glass slide via plasma treatment. The result was a glass-PD MS device with a 3 cm diameter well for lymph node culture (FIG. 1). Glass capillary tubes were pulled on a pipette puller and beveled to define the outer glass needle diameter and bevel angle. A one millimeter biopsy punch was used to make holes for glass needle insertion into the PDMS housing. The ends of the glass needle were connected to low compliance tygon tubing that connects to syringe pumps or eluent collection tubes. Cannulation of lymph node vessels was non-trivial. The glass needle was used to cannulate the vessel under a stereoscope and a suture (10-0) was used to secure the vessel to the tube. In the case of the lymphatics, as they were a low-pressure system with low flow rates, a simple square knot was sufficient to secure the afferent and efferent lymphatic vessels (FIG. 2). However, in higher pressure and flow rate connections, a modified finger-trap suture was used for the vascular connections to the glass needle. Whereas all afferent lymphatic vessels can be cannulated, the viability of cannulating and perfusing fewer of them, suturing the remaining afferent lymphatic vessels closed were determined. An important focus and outcome of the development of this hLNChip model was the design of simple and reliable connections between organ vessels and microfluidic connections to enable dissemination of these techniques to other investigators.

For lymph node culture, the organ was perfused and cultured in a complete medium bath (FIG. 1). Complete medium consisted of RPMI-1640 supplemented with 10% FBS, 1% L-glutamine, 1% Pen/Strep, 50 μM 2-mercaptorethanol, 1 mM pyruvate, 1% nonessential amino acids, and 20 mM HEPES. The lymphatic and vascular systems were perfused with a complete medium. Heparinized plasma or patient-matched heparinized whole blood in the vascular circulation was tested. Normal lymph transport rates are in the range of approximately 166.7 μm/s to 700 μm/s, depending on factors such as anatomical location, age, sex, and BMI. Blood flow rate also depends on multiple parameters, however using percentage of cardiac output to reach a lymph node, the media flow rate needed to model blood in the lymph node was estimated at approximately 8 μUs. Volumetric flow rates for lymphatic and vascular perfusion were calculated after measurement of vessel diameter. Reproduction of the pulsatile flows in the vascular compartment was tested for successful maintenance of ex vivo organ culture in the hLNChip platform.

The efferent lymph and venous flows were collected and sampled at various intervals as described below. As a demonstration of feasibility, a human lymph node was successfully cultured in a prototype hLNChip for 48 hours.

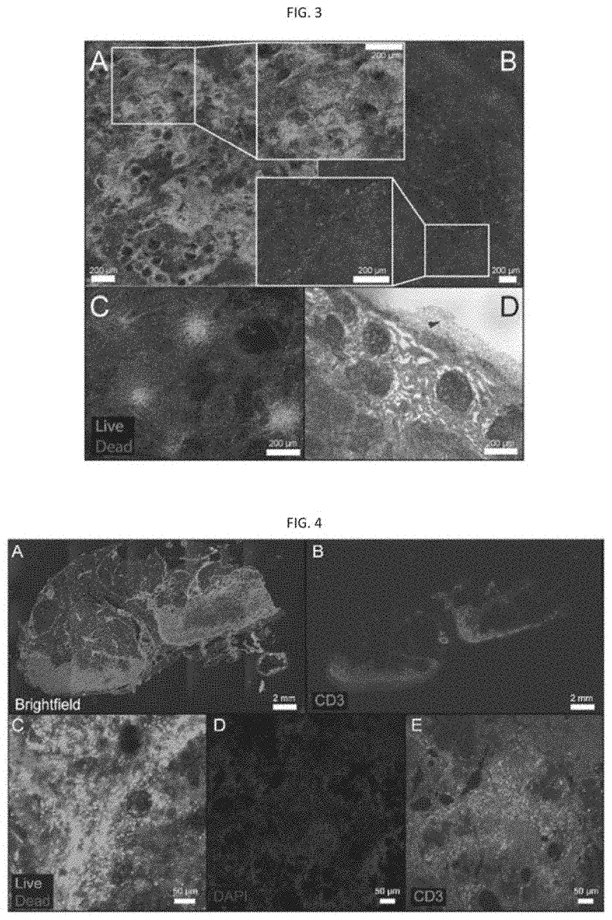

As the afferent lymphatic vessels are more fragile and less patent than the lymph node vasculature, for feasibility a lymph node was cannulated and perfused using only an afferent lymphatic vessel and culture for 24 hours compared to a non-perfused lymph node cultured for the same duration (FIG. 3). Following culture, the lymph node was cut along a sagittal plane with a scalpel and incubated the cut face with reagents from a live-dead kit. A human lymph node cultured in a medium bath without perfusion (control) resulted in widespread cell death throughout the lymph node as expected (FIG. 3B). However, a perfused human lymph node (FIG. 3A, 3C, 3D) demonstrates significant cell viability with modest staining for dead cells. These results are expected to be even further improved if the vasculature is perfused along with the afferent lymphatics.

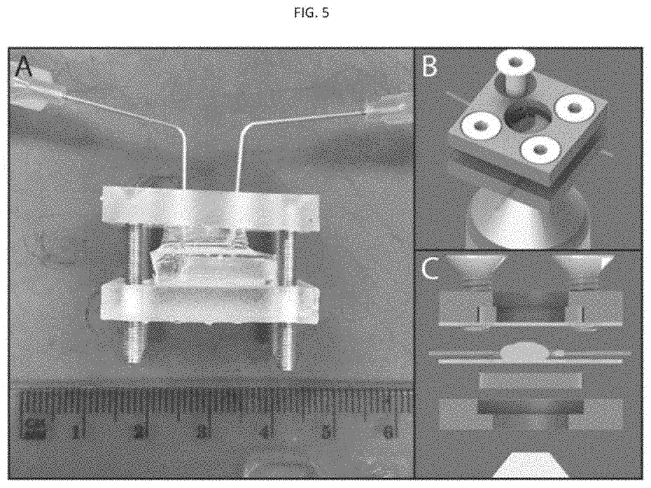

Following 48 hours of culture (vessels cannulated and perfused) as described in FIG. 3, lymph nodes were serially sectioned and imaged using brightfield, and immunofluorescence methods. FIG. 4 shows T cell depletion following 48 hours of perfused culture evident in A) brightfield and B) CD3 immunostained histological LN sections. Imaging LN lobule with C) Live/Dead D) DAPI and E) CD3 IHC, demonstrate viability of remaining T lymphocytes following 48 hours of ex vivo culture. Additionally, these data demonstrate the loss of lymphocytes from the lymph node, as is expected and normal in a functioning organ.

Assaying lymph node health—longitudinal measurements: Longitudinal measurements of cell health will be conducted using two main methods: 1) assaying perfusate and eluent fluid into and out of the lymph node and 2) fine needle biopsy/aspiration. If the lymph node is healthy, cells are expected to be detected in the efferent compartments of both the lymphatic and vascular compartments. Flow cytometry and markers will be used for various immune cells including CD4 and CD8 for T-lymphocytes and CD20 for B-lymphocytes to quantify the cells that are leaving the lymph node. Subsequently, their health will be determined using an Annexin V/PI assay to quantify cells that are alive, apoptotic, or necrotic. Macrophage function within the lymph node will be tested indirectly by introducing 2 mg/ml of bovine serum albumin (BSA) into the afferent lymphatic network and quantifying efferent lymphatic BSA concentration after 2 hours using the Bradford protein assay. Macrophages are expected to take up the albumin resulting in lower concentrations of BSA in the efferent fluid. Additionally, efferent fluid will be sampled via ELISA for ATPlite (Perkin Elmer), cyclophilin A and chromatin protein high-mobility group B (HMGB1) as markers of necrosis and soluble TNFR-1, soluble TRAILR-2, and soluble Fas as markers of apoptosis. To determine more localized cell health within the lymph node, fine needle aspiration will be used to retrieve small cell aspirates from the organ without compromising the entire lymph node. Aspirates will be assessed for apoptotic and necrotic factors using ELISA.

Assaying lymph node health—end-point measurements: To confirm data obtained in longitudinal measurements, endpoint experiments will be performed with lymph node culture being stopped at discrete time points (24 hrs, 48 hrs, 72 hrs, 5 days, 10 days, 21 days) and lymph node health comprehensively assessed. For a few lymph nodes, proliferation will be assessed by perfusing both the vascular and lymphatic circulations with EdU (EdU assay) for four hours. Subsequently, the organ will be fixed, cut with a scalpel, EdU developed and imaged to quantify areas of proliferation within the lymph node. Following culture, additional lymph nodes will be cut along a sagittal plane with a scalpel with half of the organ dissociated for the collection of cells and the other half processed for histology/immunohistochemistry (IHC). Isolated cells will undergo live-dead staining using a viability/cytotoxicity kit and quantified via flow cytometry. Additionally, live, apoptotic, and necrotic populations of immune, reticular, stromal, and vascular cells will be quantified using Annexin-V/PI and the respective cellular markers for flow cytometry. Histology/IHC methods are described below for the quantification of lymph node architecture.

Verification and quantification of lymph node architecture: Similar to assays investigating lymph node cellular health, two approaches will be used to quantify and monitor lymph node architecture over culture. For longitudinal measurements, lymph nodes will be imaged using a Bruker SkyScan 1276 in vivo microCT. The hLNChip device is designed to be MRI and CT compatible. For microCT, scans will be taken using short scan X-ray settings of 70 kV, 90 mA, 3000 ms exposure time, with 400 views, and 0.028 mm/pixel resolution. To validate the longitudinal scans and confirm distinguishable vascular tissue architecture, some lymph nodes will be fixed following the scan and perfuse with 1% 12 contrast solution for 14 hours to stain the tissue and reimage with microCT. To validate that the microCT scans reflect correct tissue architecture, some lymph nodes post scan will be sacrifice and microCT will be compared to histological sections of the same lymph node. These end-point histological assays will be complemented by IHC assays to determine the spatial localization of relevant cell populations including immune cells (CD4, CD8, CD20, CD11c), reticular cells, stromal cells, and vasculature (PECAM1, PNad).

### Example 2. Intravital Window Culture Device

Intravital imaging to visualize cells has been done in mice because it is an abundant animal model with a thin/near transparent capsule, but this hasn't been done with larger animals/humans. To visualize lymphocytes or other components or structures in an ex vivo lymph node in situ, an intravital window culture device (FIG. 5) has been designed. The lymph node capsule can be cut and a part of it is removed. A glass coverslip coated with a transparent gasket material, for example, an elastomeric polymer (e.g., poly dimethylsiloxane (PDMS)) that is spin coated on the coverslip can be used to seal the cut surface of the lymph node for imaging on a microscope. The glass coverslip is clamped to the lymph node, by securing the lymph node between to places clamped with screws. A) Side view of device prototype and CAD rendering B) isometric view and C) exploded view.

The intravital window culture device may include: 1) a culture of an ex vivo lymph node having part of an endogenous capsule cut off, 2) a glass slide or coverslip coated with a transparent deformable gasket material (e.g., PDMS, a hydrogel), and 3) a system to keep the ex vivo lymph node-gasket-glass cover slip assembly together to seal the cut area of the ex vivo lymph node capsule to prevent leaks. Imaging may occur through the glass coverslip, and optionally gasket, into the ex vivo lymph node through the cut capsule during culture. The intravital window culture device may be used to visualize cells (e.g., lymphocytes) and other structures in, and monitor behavior changes within an ex vivo lymph node.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. An ex vivo lymph node, comprising an intact lobule in a chamber connected to an afferent lymphatic vessel and an efferent lymphatic vessel, wherein the intact lobule retains viable endogenous cells in the intact lobule in vivo, and an endogenous artery and an endogenous vein each attached to the intact lobule in vivo when the intact lobule is removed from a donor, and wherein the intact lobule is perfused with a lymphatic fluid into the chamber via the afferent lymphatic vessel and out of the chamber via the efferent lymphatic vessel and perfused with a vascular fluid into the intact lobule via the endogenous artery and out of the intact lobule via the endogenous vein.

2. The ex vivo lymph node of claim 1, further comprising an endogenous capsule enclosing the chamber and encompassing the intact lobule, wherein the afferent lymphatic vessel is an endogenous afferent lymphatic vessel attached to the endogenous capsule, and the efferent lymphatic vessel is an endogenous efferent lymphatic vessel attached to the endogenous capsule in vivo when the intact lobule is removed from the donor.

3. The ex vivo lymph node of claim 2, further comprising an endogenous sinus adjacent to the intact lobule and encompassed by the endogenous capsule in vivo when the intact lobule is removed from the donor.

4. The ex vivo lymph node of claim 1, wherein at least 80% of the endogenous cells remain viable for at least 24 hours after the intact lobule is removed from the donor.

5. The ex vivo lymph node of claim 1, wherein the intact lobule is perfused with the lymphatic fluid and the vascular fluid for at least 30 minutes.

6. The ex vivo lymph node of claim 1, wherein the lymphatic fluid comprises an inlet lymphatic fluid flowing into the chamber from a lymphatic fluid reservoir via the afferent lymphatic vessel and an outlet lymphatic fluid flowing out of the chamber into an efferent collection via the efferent lymphatic vessel, wherein the inlet lymphatic fluid and the outlet lymphatic fluid have different components.

7. The ex vivo lymph node of claim 1, wherein the vascular fluid comprises an inlet vascular fluid flowing into the intact lobule from a vascular fluid reservoir via the endogenous artery and an outlet vascular fluid flowing out of the lobule into a venous collection via the endogenous vein, wherein the inlet vascular fluid and the outlet vascular fluid have different components.

8. The ex vivo lymph node of claim 1, wherein the lymphatic fluid flows under control by a lymphatic inlet pressure.

9. The ex vivo lymph node of claim 1, wherein the vascular fluid flows under control by a vascular inlet pressure.

10. The ex vivo lymph node of claim 1, wherein the lymphatic fluid and the vascular fluid flow independently.

11. The ex vivo lymph node of claim 1, wherein at least one of the afferent lymphatic vessel, the efferent lymphatic vessel, the endogenous artery and the endogenous vein is connected directly to a cannulation device.

12. The ex vivo lymph node of claim 1, wherein an additional endogenous artery is attached to the intact lobule and closed off.

13. The ex vivo lymph node of claim 1, wherein the intact lobule is cultured.

14. The ex vivo lymph node of claim 1, wherein the endogenous cells comprise B lymphocytes, T lymphocytes, macrophages, dendritic cells, follicular dendritic cells, red blood cells or a combination thereof.

15-16. (canceled)

17. A method for preparing an ex vivo lymph node, comprising:
(a) providing an intact lobule in a chamber, wherein the intact lobule retains viable endogenous cells in the intact lobule, and an endogenous artery and an endogenous vein each attached to the intact lobule in vivo when the intact lobule is removed from a donor, wherein the chamber is connected to an afferent lymphatic vessel and an efferent lymphatic vessel;
(b) perfusing the intact lobule with a lymphatic fluid flowing into the chamber via the afferent lymphatic vessel and out of the chamber via the efferent lymphatic vessel; and
(c) perfusing the intact lobule with a vascular fluid flowing into the intact lobule via the endogenous artery and out of the intact lobule via the endogenous vein.

18-29. (canceled)

30. An ex vivo lymph node prepared according to the method of claim 17.

31. A method for screening for an agent capable of changing the ex vivo lymph node of claim 1, comprising:
(a) exposing the ex vivo lymph node to a test agent; and
(b) monitoring a behavior of the intact lobule before and after the exposure, wherein a change in the behavior after the exposure indicates that the test agent is capable of changing the ex vivo lymph node.

32-36. (canceled)

37. A method for producing T lymphocytes, comprising exposing the ex vivo lymph node of claim 1 to a cognate antigen under conditions suitable for proliferation of cognate antigen specific T lymphocytes, whereby the number of the cognate antigen specific T lymphocytes increases at least three times after the exposure.

38. (canceled)

39. A method for producing B lymphocytes, comprising exposing the ex vivo lymph node of claim 1 to an antigen under conditions suitable for proliferation of antigen specific B lymphocytes, whereby the number of the antigen specific B lymphocytes increases at least three times after the exposure.

40. (canceled)

41. A method for determining immunoreactivity of the ex vivo lymph node of claim 1, comprising exposing the ex vivo lymph node to an antigen under conditions suitable for proliferation of antigen specific lymphocytes, and monitoring a behavior of the intact lobule before and after the exposure, wherein a change in the behavior upon exposure indicates that the ex vivo lymph node is immunoreactive to the antigen.

42-44. (canceled)
